(19) United States
(12) Patent Application Publication
(10) Pub. No.: US 2004/0057913 A1
(43) Pub. Date: Mar. 25, 2004

(54) Antioxidant and skin preparations for external use

(76) Inventors: Toshiro Sone, Tokyo (JP); Katsuyoshi Chiba, Tokyo (JP); Ryoko Izuka, Tokyo (JP); Tomoko Hanamizu, Tokyo (JP); Hirosi Kono, Tokyo (JP); Masahiko Iha, Gushikawa-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/276,330

(22) PCT Filed: Apr. 12, 2001
(86) PCT No.: PCT/JP01/03148

(30) Foreign Application Priority Data
May 26, 2000 (JP) 2000-155924

(51) Int. Cl. A61K 7/42; A61K 35/78

(52) U.S. Cl. 424/59; 424/725

(57) ABSTRACT

An antioxidant containing as the active ingredient a Mozuku extract; and skin preparations for external use which contain this antioxidant. These preparations, which are suitable in treating inflammation, are highly efficacious, less expensive and can be applied everyday.

### FIELD OF THE INVENTION

The present invention relates to an antioxidant and a skin preparation for external use. More particularly, the present invention relates to an antioxidant containing Mozuku extract as an active ingredient that exhibits an excellent antioxidizing and anti-inflammatory effect in response to ultraviolet rays or a stimulator and to a skin preparation for external use such as a cosmetic containing the antioxidant that exhibits an excellent sensation during use.

### DESCRIPTION OF BACKGROUND ART

Active oxygen is produced when the skin is exposed to ultraviolet rays. This is believed to be one of the causes of the inflammatory reaction that occurs thereafter. Conventionally, sunscreens have been used to prevent the inflammatory reaction because they exhibit an excellent effect. However, due to problems in sensation during use such as touch sensation and limitations to physical durability properties such as anti-chaffing and anti-sweat properties, these sunscreens have not been satisfactory in their capabilities of exhibiting a continuous preventive effect. A preparation that can prevent injury after exposure to ultraviolet rays has also been desired.

Steroidal anti-inflammatory agents have been used to reduce inflammation resulting from ultraviolet rays. Even though the curative effect of these agents has been confirmed, the steroids target many internal organs and result in serious side effects. As an alternative, various non-steroidal anti-inflammatory agents are used. However, these agents cannot easily be used because of a problem of causing stomach ulcers due to suppression of prostaglandin.

Also, there have been many reports regarding plant extracts. For example, glycyrrhiza extract, scutellaria root extract, chamomile extract (Japanese Patent Application Laid-open No. 1997/157172), spatholobus suberectus root extract (Japanese Patent Application Laid-open No. 1996/26965), astragalus root extract (Japanese Patent Application Laid-open No. 1996/73325), and the like have been used to reduce inflammation caused by ultraviolet rays. Their effect, however, is not satisfactory.

On the other hand, there are many reports on skin preparations for external use and cosmetic compositions containing seaweed extract, especially Mozuku of Phaeophyceae. These preparations possess a whitening effect (Japanese Patent Application Laid-open No. 1991/251514), anti-wrinkle effect (Japanese Patent Application Laid-open No. 1997/268119), and moisturizing effect (Japanese Patent Publication No. 1996/766). Also, it is known that fucoidan, the active component of Mozuku, exhibits an anti-allergic effect (Japanese Patent Application Laid-open No. 1999/21247) and a moisturizing effect (Japanese Patent Application Laid-open No. 1989/31707). These reports, however, mention neither the antioxidation effect nor the effect of preventing or curing inflammation resulting from ultraviolet rays.

From the viewpoint of side effects, pharmaceutical products presently used for the treatment of inflammation resulting from the production of active oxygen when the skin is exposed to ultraviolet rays cannot always be confidently used. Also, there have been very few plant extracts that could be used satisfactorily from the viewpoint of effectiveness and cost. Therefore, a low priced highly effective product that can be used on a day-to-day basis has been desired.

Accordingly, the object of the present invention is to provide an antioxidant effective in preventing and curing inflammation resulting from ultraviolet rays, for example, as a material that can be safely used on a day-to-day basis, and to provide a skin preparation for external use comprising this antioxidant.

### DISCLOSURE OF THE INVENTION

In order to achieve the above object, the present inventors examined the antioxidation activities of various compounds and have discovered that the extract of Mozuku possesses an excellent antioxidation and anti-inflammation effect against ultraviolet rays or stimulators. The inventors have also discovered that the extract of Mozuku possesses an excellent sensation during use, thereby completing the present invention.

Specifically, the present invention provides an antioxidant comprising the extract of Mozuku as an active component.

The present invention also provides a skin preparation for external use comprising the above antioxidant.

### BRIEF DESCRIPTION OF THE DRAWINGS

FIG. 1 shows the test results of the radical scavenging effect of various kinds of seaweed extracts at various concentrations.

FIG. 2 shows the test results of the SOD-like activity of Mozuku extract at various concentrations.

FIG. 3 shows the test results of the TNFα secretion suppressing activity of Mozuku extract after ultraviolet irradiation.

FIG. 4 shows the test results of the mouse ear edema suppressing activity of Mozuku extract.

### DETAILED DESCRIPTION OF THE INVENTION AND PREFERRED EMBODIMENT

The extract obtained from Mozuku (hereinafter referred to as “Mozuku extract”) used as the active component in the antioxidant of the present invention is extracted from the seaweed Mozuku using a suitable solvent such as water or alcohol.

As Mozuku, for example, Tinocladia crassa Kylyn, Hydrilla verticillata, Cladosiphon okamuranus Tokida, Analipus japonicus Wynnu, Nemacystus decipiens Kuckuck, Heterosaundersella hattoriana Tokida, Sphaerotrichia divaricata Kylin, Myriogloea simplex Inagaki, and the like can be used with cultivated Cladosiphon okamuranus Tokida being preferable from the effect and economic viewpoints. Dried, salt-cured, or frozen Cladosiphon okamuranus Tokida may be used.

The extract of Mozuku can be obtained by adding a suitable amount of solvent to the above Mozuku raw material at room temperature or under heating and allowing the raw material to soak. Examples of the solvent used include, in addition to water, ethanol, methanol, polyhydric alcohols such as propylene glycol, 1,3-butylene glycol, and glycerol, as well as mixtures of these alcohols.

As the method for manufacturing the Mozuku extract, there are many methods that have been reported (Japanese Patent Publication No. 1996/766, Japanese Patent Application Laid-open Nos. 1998/245334 and 1999/1437, and the like) Any one of these methods may be used in the present invention. The following method can be given as a specific example for manufacturing Mozuku extract preferably used in the present invention. A solvent such as water or an acidic aqueous solution (pH 2.5-3.0) is added to the whole seaweed of frozen Cladosiphon okamuranus Tokida. The mixture is repeatedly extracted with heating, filtered, and desalted to obtain Mozuku extract. The obtained extract is light yellow to light brown and has a slight peculiar odor. The obtained Mozuku extract is concentrated under reduced pressure at a temperature of 50° C. or less. The concentrate maybe frozen or spray-dried to obtain extract powder. If necessary, the extract may be fractionated using an appropriate separation means such as gel filtration, electrodialysis, silica gel column chromatography, HPLC, or the like to obtain fractions with high activity.

The Mozuku extract obtained in this manner may be independently used as an antioxidant for preventing or curing inflammation due to ultraviolet radiation. More preferably, however, the Mozuku extract is used as an antioxidant in combination with a suitable known pharmaceutical carrier.

The antioxidant containing the Mozuku extract obtained in this manner is a material derived from an edible seaweed and, therefore, is completely free of anything harmful. The antioxidant can be combined with a suitable known base for external preparation and used as a skin preparation for external use such as a cosmetic, drug, quasi-drug, or the like. The amount of antioxidant (the amount of the solid Mozuku extract) is 0.0001-30 wt % (hereinafter indicated as “%”), and particularly preferably 0.001-10% of the total amount of the preparation for external use.

The skin preparation for external use can be prepared by formulating, as required, additives commonly used in skin preparations for external use, such as other pharmaceutical components, water, alcohols, humectants, oil components, amphipathic compounds, thickeners, UV absorbers, chelating agents, pH controllers, antiseptic agents, coloring matters, perfumes, and the like, in addition to the antioxidant containing the Mozuku extract. The skin preparation for external use of the present invention may contain the above essential components in a lamellar structure or the preparation may be in the form of emulsion.

Analgesics, antiphlogistics, bactericides, antiseptics, and the like can be given as other pharmaceutical components used in the preparation of the skin preparation for external use. Specific examples of analgesics and antiphlogistics include methyl salicylate, camphor, tannic acid, mefenamic acid, ibuprofen, benzydamine, hydrocortisone, and the like. As specific examples of bactericides and antiseptics, salicylic acid, ichthammol, chlorohexidine, gentamicin, and the like can be given. As humectants, hyaluronic acid, lactobacillus culture broth, polyhydric alcohols such as glycerol, propylene glycol, 1,3-butylene glycol, sorbitol, polyglycerol, polyethylene glycol, and dipropylene glycol, NMF components such as amino acids, sodium lactate, and sodium pyrrolidone carboxylate, water-soluble high molecular weight compounds such as collagen, mucopolysaccharides, and chondroitin sulfate, and the like can be given.

As oil components among the optional components, the following examples can be given: vegetable oils and fats such as castor oil, olive oil, cacao oil, camellia oil, coconut oil, Japan wax, jojoba oil, grape seed oil, and avocado oil; animal oils and fats such as mink oil and egg yolk oil; waxes such as beeswax, whale wax, lanolin, carnauba wax, and candelila wax; hydrocarbons such as liquid paraffin, squalane, micro-crystallin wax, ceresin wax, paraffin wax, and petroleum jelly; natural and synthetic fatty acids such as lauric acid, myristic acid, stearic acid, oleic acid, iso-stearic acid, and behenic acid; natural and synthetic higher alcohols such as cetyl alcohol, stearyl alcohol, hexyldecanol, octyl dodecanol, and lauryl alcohol; and esters such as isopropyl myristate, isopropyl palmitate, octyldodecyl myristate, octyldodecyl oleate, and cholesterol oleate.

The following compounds can be given as examples of amphipathic compounds: nonionic surfactants such as oleophillic glycerol monostearate, self-emulsifiable glycerol monostearate, polyglycerol monostearate, sorbitan monooleate, polyethylene glycol monostearate, polyoxyethylene sorbitan monooleate, polyoxyethylene cetyl ether, polyoxyethylene sterol, polyoxyethylene lanolin, polyoxyethylene bees wax, and polyoxyethylene hydrogenated castor oil; anionic surfactants such as sodium stearate, potassium palmitate, sodium cetyl sulfate, sodium lauryl phosphate, triethanolamine palmitate, polyoxyethylene sodium lauryl phosphate, and sodium N-acyl glutamate; cationic surfactants such as stearyl dimethylbenzyl ammonium chloride and stearyl trimethyl ammonium chloride; amphoteric surfactants such as alkylaminoethyl glycine chloride and lecithin; and the like.

As thickners, sodium alginate, xanthan gum, aluminum silicate, quince extract, naturally occurring polymers such as traganth gum and starch, half-synthetic polymers such as methylcellulose, hydroxyethylcellulose, carboxymethylcellulose, soluble starch, and cationized cellulose, synthetic polymers such as carboxy vinyl polymer and polyvinyl alcohol, and the like can be given. As UV absorbers or dispersion agents, 2-hydroxy-4-methoxybenzophenone, octyldimethyl p-aminobenzoate, ethylhexyl p-methoxycynamate, titanium oxide, kaolin, talc, and the like can be given. As vitamins, Vitamin A, vitamin B, vitamin C, vitamin D, vitamin E, vitamin F, vitamin K, vitamin P, vitamin U, carnitine, ferulic acid, γ-oryzanol, α-lipoic acid, orotic acid, derivatives of these vitamins, and the like can be given. As amino acids, glycine, alanine, valine, leucine, isoleucine, serine, threonine, phenylalanine, thyrosin, tryptophan, cystine, cysteine, methionine, proline, hydroxyproline, aspartic acid, glutamic acid, arginine, histidine, lysine, derivatives of these vitamins, and the like can be given.

As antiseptic agents, benzoate, salicylate, sorbate, dehydroacetate, p-oxybenzoate, 2,4,4′-trichloro-2′-hydroxydiphenyl ether, 3,4,4′-trichlorocarboanilide, benzalkonium chloride, hinokitiol, resorcin, ethanol, and the like can be given.

The skin preparation for external use of the present invention, comprising cosmetics, drugs, quasi-drugs, and the like as mentioned above, may be prepared in various forms such as a face lotion, milky lotion, moisturizing cream, cleansing cream, massage cream, face cream, pack, beauty solution, shampoo, body shampoo, body lotion, solid soap, and the like by conventional methods.

### EXAMPLES

The present invention will be described in more detail by way of examples, reference examples, and test examples which should not be construed as limiting the present invention.

### Reference Example 1

Preparation of Seaweed Extracts

(1) Preparation of Mozuku Extract

2 l of water was added to 1 kg of frozen stored Cladosiphon okamuranus Tokida and the mixture was adjusted to pH 3.0 by the addition of 2N HCl aqueous solution. The mixture was heated at 90° C. for one hour while stirring to extract the seaweed essence. Immediately after extraction, the extract was neutralized to pH 5.0 with 2N sodium hydroxide and cooled. After removing impurities by filtration, the extract was desalted by electrodialysis and concentrated under reduced pressure to a Brix of 3% or more. The concentrated extract was freeze-dried to obtain about 20 g of Mozuku extract.

(2) Preparation of Laminariales Extract

2 l of water was added to 1 kg of frozen stored Laminaria japonica and the mixture was adjusted to pH 3.0 by the addition of 2N HCl aqueous solution. The mixture was heated at 90° C. for one hour while stirring to extract the Laminariales essence. Immediately after extraction, the extract was neutralized to pH 5.0 with 2N sodium hydroxide and cooled. After removing impurities by filtration, the extract was desalted by electrodialysis and concentrated under reduced pressure to a Brix of 3% or more. The concentrated extract was freeze-dried to obtain about 20 g of Laminariales extract.

(3) Preparation of Fucales Extract

2 l of water was added to 1 kg of frozen stored Pelvetia wrightii and the mixture was adjusted to pH 3.0 by the addition of 2N HCl aqueous solution. The mixture was heated at 90° C. for one hour while stirring to extract the Fucales essence. Immediately after extraction, the extract was neutralized to pH 5.0 with 2N sodium hydroxide and cooled. After removing impurities by filtration, the extract was desalted by electrodialysis and concentrated under reduced pressure to a Brix of 3% or more. The obtained concentrated extract was freeze-dried to obtain about 20 g of Fucales extract.

### Test Example 1

Radical Scavenging Effect

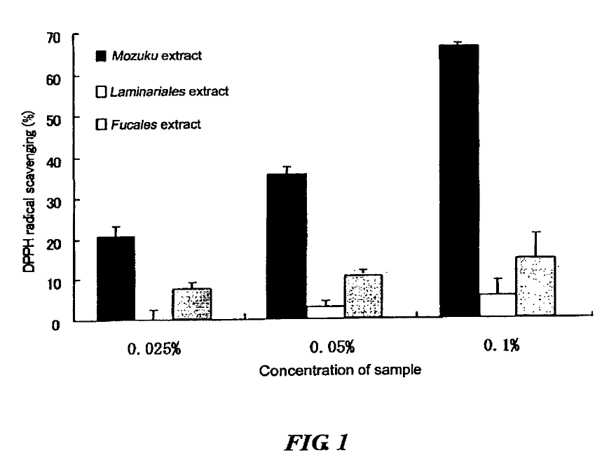

The radical scavenging effect of various seaweed extracts was examined as follows. The Mozuku extract, Laminariales extract, and Fucales extract were used as test specimens. 100 μl of a 20% ethanol solution of the test specimen, 50 μl of 200 mM acetic acid buffer solution (pH 5.5), and 100 μl of 250 μM 2,2-diphenyl-1-picrylhydrazyl picrylhydrazyl (DPPH; ethanol solution) were charged into wells and incubated at 30° C. for 30 minutes to measure the absorbance at a wavelength of OD₅₁₅ nm. The radical scavenging rate (%) of DPPH was calculated from the following formula. A sample with a solvent added instead of the test specimen was used as a control. The results are shown in FIG. 1.

DPPH radical scavenging rate (%)={(A−B)/A}×100

A: The OD₅₁₅ value of the control

B: The OD₅₁₅ value of the test specimen

FIG. 1 clearly shows the DPPH radical scavenging effect of Mozuku extract. In addition, the Mozuku extract was shown to exhibit a better antioxidizing effect than Laminariales extract and Fucales extract.

### Test Example 2

Superoxide Anion Diminishing Activity (SOD-Like Activity)

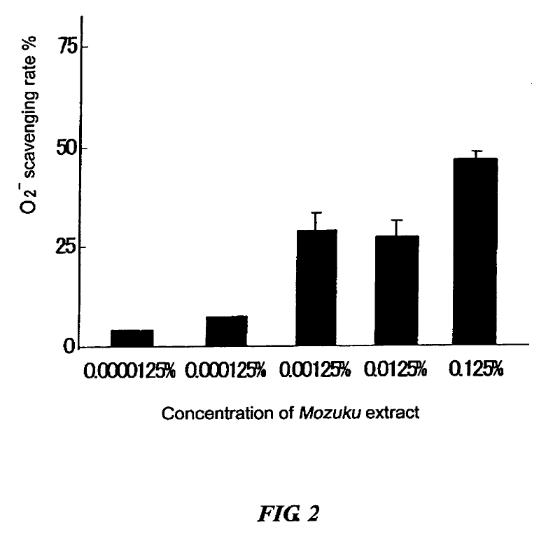

The SOD-like activity of Mozuku extract was measured according to the SOD-test Wako (Wako Pure Chemical Industries, Ltd.) based on the principle of the nitro blue tetrazolium (NTB) reduction method using a 96-well microplate. Specifically, 10 μl of a solution of Mozuku extract with a prescribed concentration in 20% ethanol was added as a test specimen to 95 μl of a coloring reagent containing 0.24 mM NBT and 0.4 mM xanthine. After further addition of 95 μl of an enzyme (xanthine oxidase) solution, the mixture was incubated for 30 minutes at 37° C. The enzyme reaction was terminated with the addition of 100 μl of a reaction termination solution (69 mM sodium dodecyl sulfate) to measure the absorbance at a wavelength of OD₅₆₂ nm using a micro-plate reader. A sample prepared by adding purified water instead of the enzyme (a blank) and a solvent instead of the test specimen was used as a control. The superoxide anion (O₂⁻) scavenging rate was calculated according to the following formula. The results are shown in FIG. 2.

O₂⁻ scavenging rate (%)={(A−B)/A}×100

A: The OD₅₆₂ value of the control

B: The OD₅₆₂ value of the test specimen

The results of FIG. 2 have made it clear that the ° 2-scavenging rate of Mozuku extract is weak even at a low concentration, demonstrating superior scavenging activity of the Mozuku extract.

### Test Example 3

TNFα Secretion Controlling Activity on Human Keratinocyte Irradiated with UV Light:

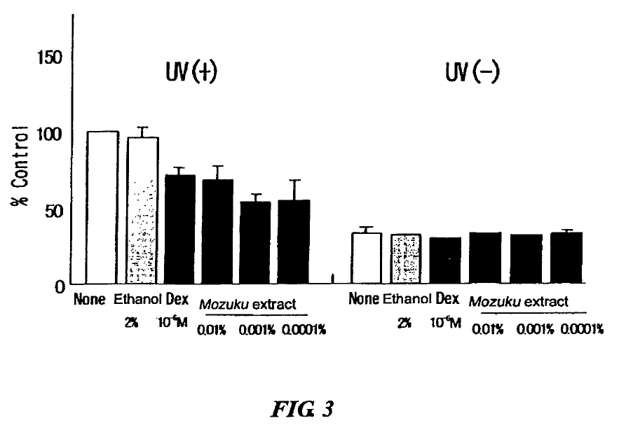

TNFα secretion controlling activity of the Mozuku extract effect on human keratinocyte irradiated with UV light was examined using normal human keratinocytes transformed with SV40 virus T-antigen as cells. The cells were cultured in a 5% CO₂ incubator at 37° C. using HuMedia-KG2 (Kurabo Industries, Ltd.) as a culture medium. For differentiation induction, the cells were cultured for 24 hours by replacing the medium with a high concentration calcium culture medium (1 mM CaCl₂ added). The cells were irradiated with UVB for 25 seconds in PBS at a dose of 200 J/m² using a 312 nm UV lamp (VILBER LOURMAT Co.). Then, the cells were cultured in a usual culture medium or with the addition of a sample. The amount of TNFα in the culture medium supernatant liquid at 24 hours after irradiation was measured using a high sensitive ELISA kit (BIO SOURCE). The results are shown in FIG. 3.

FIG. 3 shows that the Mozuku extract controlled secretion of TNFα due to irradiation of UV rays at a concentration of 0.01-0.0001%. The effect was equivalent to or more than the effect obtained by dexamethasone (Dex), a 10⁻⁶ M anti-inflammatory steroid, used as a positive control. No toxicity to the cells was observed. In this manner, the Mozuku extract was found to control excessive secretion of TNFα, which is known as an inflammatory cytokine, due to irradiation with UVB.

### Test Example 4

Mouse Ear Swelling Suppressing Activity

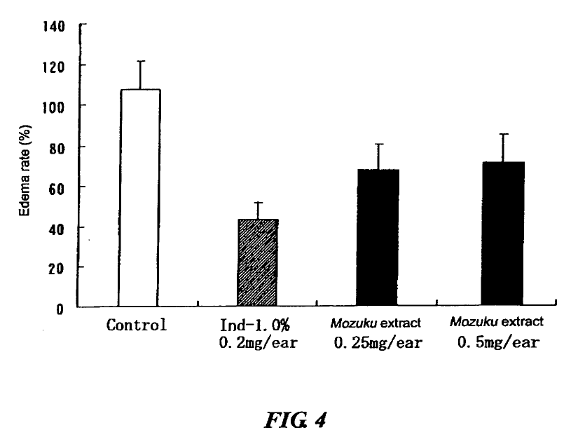

Mouse ear swelling suppressing activity of the Mozuku extract was examined as follows. The thickness of the ear of an ICR male mouse (age: 5 weeks) was measured using a thickness gauge (Digimatic Indicator 543, manufactured by Mitsutoyo Co., Ltd.). The Mozuku extract was applied to the ear in the amount of 0.25 mg/ear or 0.5 mg/ear. As a comparative test, an indomethacin solution, prepared by dissolving indomethacin at a concentration of 1.0% in 70% acetone, was applied to the mouse ear in the amount of 0.2 mg/ear. 30 minutes after the application, 10 μl of a 40 μl/ml solution of 12-O-tetradecanoylphorbol-13-acetate (TPA) in 70% acetone was applied to the in and out sides of the mouse ear to induce a skin reaction. The thickness of the ear skin was measured after 4 hours at which the value of the swelling became the maximum. The ratio of the measured thickness to the thickness of the ear skin before the edema induction was calculated as the ratio of edema. The thickness of both the right and left ear skins was measured and the values were averaged to determine the ear skin thickness of the mouse. The results are shown in FIG. 4.

It can be seen from FIG. 4 that edema was induced by applying TPA to the ear skin of the mouse, increasing the ear skin thickness two times or more. A remarkable edema suppressing effect was exhibited when indomethacin or Mozuku extract is applied 30 minutes before the TPA application. The same results were seen when the specimen was applied simultaneously with TPA. In this manner, the Mozuku extract exhibits a superior anti-inflammatory action not only against UV rays but also against a chemical stimulant.

### Example 1

A face lotion of the following composition was prepared.

Formulation:

| (1) | Ethanol | 5.0 | (wt %) |
|---|---|---|---|
| (2) | 1,3-Butylene glycol | 2.0 | |
| (3) | Hyaluronic acid | 0.2 | |
| (4) | Polyoxyethylene hydrogenated castor oil | 0.05 | |
| (5) | Methyl p-oxybenzoate | 0.1 | |
| (6) | Perfume | 0.1 | |
| (7) | Mozuku extract (solid components) | 0.01 | |
| (8) | Distilled water | Balance | |
| | Total | 100.00 | |

Method of Preparation:

Components (3) and (7) were dissolved in component (8). Then, components (1), (2), (4), (5), and (6) were added to the solution and the mixture was sufficiently stirred to obtain a face lotion.

### Example 2

A milky lotion of the following composition was prepared.

Formulation:

| (1) | Stearic acid | 2.0 | (wt %) |
|---|---|---|---|
| (2) | Liquid paraffin | 5.0 | |
| (3) | Squalane | 2.0 | |
| (4) | Sorbitan monostearate | 1.5 | |
| (5) | Polyoxyethylene(20) sorbitan monostearate | 2.0 | |
| (6) | Butyl p-oxybenzoate | 0.05 | |
| (7) | Glycerol | 2.0 | |
| (9) | Methyl p-oxybenzoate | 0.1 | |
| (9) | Perfume | 0.15 | |
| (10) | Mozuku extract (solid components) | 0.03 | |
| (11) | Distilled water | Balance | |
| | Total | 100.00 | |

Method of Preparation:

Components (7), (8), and (10) were added to component (11). Then, components (1)-(6) were added to the mixture and emulsified at 80° C. After the addition of component (9), the emulsion was allowed to cool to room temperature to obtain a milky lotion.

### Example 3

A cream of the following composition was prepared.

Formulation:

| (1) | Liquid paraffin | 23.0 | (wt %) |
|---|---|---|---|
| (2) | Petroleum jelly | 7.0 | |
| (3) | Cetanol | 1.0 | |
| (4) | Stearic acid | 2.0 | |
| (5) | Beeswax | 2.0 | |
| (6) | Sorbitan monostearate | 3.5 | |
| (7) | Polyoxyethylene(20) sorbitan monostearate | 2.5 | |
| (8) | Butyl p-oxybenzoate | 0.05 | |
| (9) | Hyaluronic acid | 0.1 | |
| (10) | 1,3-Butylene glycol | 1.0 | |
| (11) | Methyl p-oxybenzoate | 0.1 | |
| (12) | Perfume | 0.15 | |
| (13) | Lactobacillus culture broth | 5.0 | |
| (14) | Mozuku extract (solid components) | 0.05 | |
| (15) | Distilled water | Balance | |
| | Total | 100.00 | |

Method of Preparation:

Components (10), (11), (13), and (14) were added to component (15). Component (9) was added to and dissolved in the mixture to obtain a dispersion. Then, components (1)-(8) were added to the dispersion to emulsify the mixture at 80° C. After the addition of component (12), the emulsion was allowed to cool to room temperature to obtain a cream.

### Example 4

A shampoo of the following composition was prepared.

Formulation:

| (1) | Triethanolamine lauryl sulfate | 8.5 | (wt %) |
|---|---|---|---|
| (2) | Polyoxyethylene(3) triethanolamine lauryl | 8.5 | |
| | sulfate | | |
| (3) | Coconut oil fat fatty acid diethanol amide | 3.0 | |
| (4) | Ethylene glycol monostearate | 2.0 | |
| (5) | Butyl p-oxybenzoate | 0.05 | |
| (6) | Hyaluronic acid | 0.1 | |
| (7) | 1,3-Butylene glycol | 1.0 | |
| (8) | Methyl p-oxybenzoate | 0.1 | |
| (9) | Perfume | 0.15 | |
| (10) | Lactobacillus culture broth | 3.0 | |
| (11) | Mozuku extract (solid components) | 0.5 | |
| (12) | Distilled water | Balance | |
| | Total | 100.00 | |

Method of Preparation:

Components (6), (7), (8), (10), and (11) were added to component (12). Then, components (1)-(5) were added to the mixture to emulsify at 80° C. After the addition of component (9), the emulsion was allowed to cool to room temperature to obtain a shampoo.

### INDUSTRIAL APPLICABILITY

Since the Mozuku extract has both the antioxidizing activity and the anti-inflammatory activity, inflammation can be cured or prevented by using the Mozuku extract. In addition, the Mozuku extract is highly safe and therefore can be continuously used for a long period of time.

The antioxidant containing the Mozuku extract as an active ingredient of the skin preparation for external use containing the antioxidant is effective for preventing and curing inflammation due to UV radiation and the like.

What is claimed is:

1. An antioxidant comprising extract of Mozuku as an active component.

2. The antioxidant of claim 1 having an effect of preventing and curing inflammatory due to UV light.

3. A skin preparation for external use comprising the antioxidant of claim 1 or claim 2.
